(19) United States
(12) Patent Application Publication
(10) Pub. No.: US 2011/0207931 A1
(43) Pub. Date: Aug. 25, 2011

(54) PREPARATION OF VALGANCICLOVIR AND ITS SALTS

(76) Inventors: Srinivas Katkam, Hyderabad (IN); Rajeswar Reddy Sagyam, Hyderabad (IN); Madhusudhan Reddy Ganta, Hyderabad (IN); Babu Ireni, Hyderabad (IN); Srihari Babu Korrothu, Rangareddy (IN); Raghavendar Rao Morthala, Hyderabad (IN); Narsimha Rao Pagadala, Hyderabad (IN); Veera Venkata Satyanarayana Murthy Kondepudi, East Godavari (IN)

(21) Appl. No.: 13/031,691

(22) Filed: Feb. 22, 2011

Related U.S. Application Data
(60) Provisional application No. 61/345,710, filed on May 18, 2010.

(30) Foreign Application Priority Data
Feb. 23, 2010 (IN) 469/CHE/2010

(51) Int. Cl. C07D 473/18

(52) U.S. Cl. 544/276

(57) ABSTRACT

The application relates to processes for preparing valganciclovir and pharmaceutically acceptable salts thereof, as well as intermediates for the processes. valganciclovir hydrochloride is represented by Formula II.

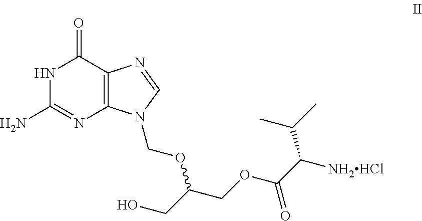

The application relates to processes for preparing valganciclovir and pharmaceutically acceptable salts thereof, as well as intermediates for the processes.

Valganciclovir (I) has a chemical name L-valine, 2-[(2-amino-1,6-dihydro-6-oxo-9H-purin-9-yl)methoxy]-3-hydroxypropyl ester. Valganciclovir hydrochloride is represented by Formula II. Valganciclovir is a mono-L-valyl ester (prodrug) of the antiviral compound ganciclovir (III). Valganciclovir hydrochloride is a cytomegalovirus (CMV) nucleoside analogue DNA polymerase inhibitor, prescribed for the treatment of CMV retinitis in patients with acquired immunodeficiency syndrome (AIDS) in adults and prevention of CMV disease in kidney, heart, and kidney-pancreas transplant patients at high risk in adults, and prevention of CMV disease in kidney and heart transplant patients at high risk in pediatric patients. The structure of valganciclovir (I) is shown below.

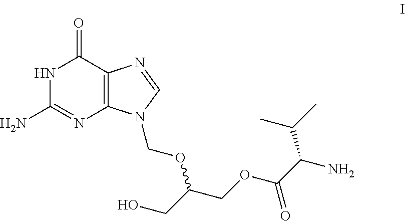

The structure of valganciclovir hydrochloride (II) is shown below.

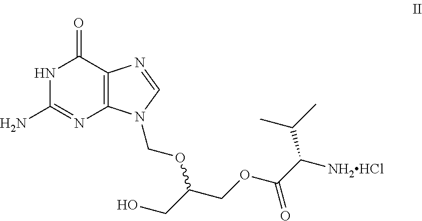

The structure of ganciclovir (III) is shown below.

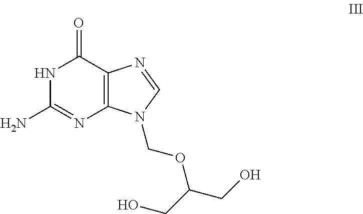

European Patent Application 0 694 547 discloses a process for partial hydrolysis of the bis-ester 2-(2-amino-1,6-dihydro-6-oxo-purin-9-yl)methoxy-1,3-propanediyl bis(L-valinate) or a salt thereof, to afford the monoester 2-(2-amino-1,6-dihydro-6-oxo-purin-9-yl)methoxy-3-hydroxy-1-propanyl-L-valinate or a pharmaceutically acceptable salt thereof. The process disclosed in Example 6 of the application involves use of preparative reverse phase HPLC column, which makes the process unsuitable for commercial scale manufacturing.

U.S. Pat. No. 5,700,936 describes processes for preparing the compound 2-(2-amino-1,6-dihydro-6-oxo-purin-9-yl)methoxy-3-hydroxy-1-propanyl-L-valinate or a pharmaceutically acceptable salt or diastereomer thereof, comprising: hydrolyzing a compound of Formula IV:

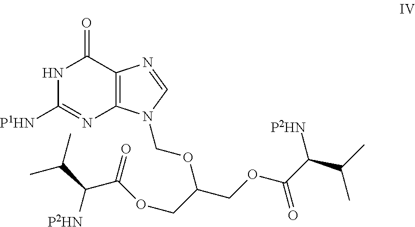

wherein P¹ is hydrogen or an amine-protecting group, and P² is an amine-protecting group; to form a compound of Formula V:

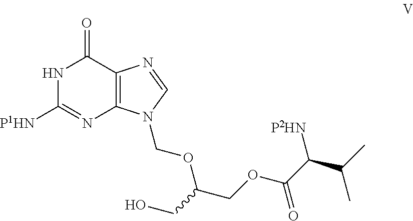

in the presence of an amine, in a nonpolar aprotic solvent; and deprotecting the compound of Formula V to 2-(2-amino-1,6-dihydro-6-oxo-purin-9-yl)methoxy-3-hydroxy-1-propanyl-L-valinate or a pharmaceutically acceptable salt thereof; optionally followed by converting 2-(2-amino-1,6-dihydro-6-oxo-purin-9-yl)methoxy-3-hydroxy-1-propanyl-L-valinate into a pharmaceutically acceptable salt thereof; or separating 2-(2-amino-1,6-dihydro-6-oxo-purin-9-yl)methoxy-3-hydroxy-propanyl-L-valinate into its (R) and (S) diastereomers.

There remains a need to provide improved processes for preparing valganciclovir and pharmaceutically acceptable salts thereof, which are simple, cost-effective, commercially viable, sustainable, environmentally friendly, and avoid multiple protection-deprotection steps.

### SUMMARY

In one aspect, the application provides methods for preparing valganciclovir and pharmaceutically acceptable salts thereof, embodiments comprising one or more of the following steps, individually or in the sequence recited:

a) reacting the compound of Formula III with a compound of Formula VI, to obtain a compound of Formula VII:

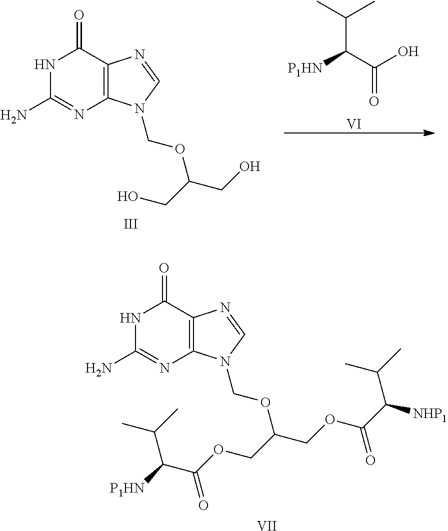

wherein P₁ is hydrogen or an amine-protecting group;

b) partially hydrolyzing a compound of Formula VII, to obtain a compound of Formula VIII:

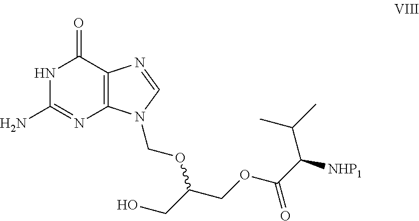

wherein P₁ is as defined above; and

c) converting a compound of Formula VIII to valganciclovir of Formula I or a salt thereof.

### DETAILED DESCRIPTION

In one aspect, the application provides methods for preparing valganciclovir and pharmaceutically acceptable salts thereof, embodiments comprising one or more of the following steps, individually or in the sequence recited:

a) reacting the compound of Formula III with a compound of Formula VI, to obtain a compound of Formula VII:

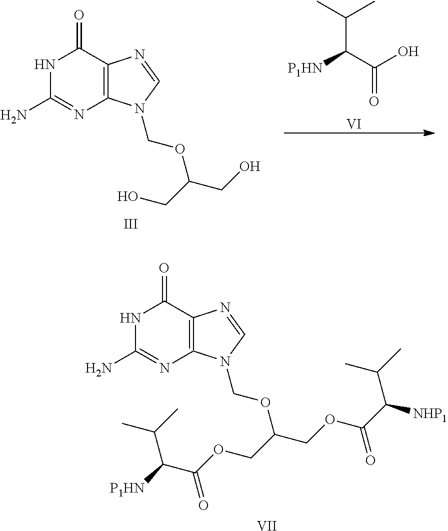

wherein P₁ is hydrogen or an amine-protecting group;

b) partially hydrolyzing a compound of Formula VII, to obtain a compound of Formula VIII:

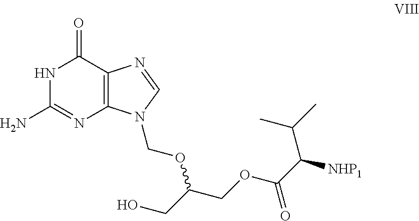

wherein P₁ is as defined above; and

c) converting a compound of Formula VIII to valganciclovir of Formula I or a salt thereof.

Step a) involves reacting the compound of Formula III with a compound of Formula VI, to obtain a compound of Formula VII. Step a) may be carried out in presence of one or more suitable base. Suitable bases, which may be used in step a) include, but are not limited to, organic bases, inorganic bases, or resins, such as, for example: aliphatic amines, e.g., triethylamine, tributylamine, N-methylmorpholine, N,N-diisopropylethylamine, N-methylpyrrolidine, or the like; aromatic amines, e.g., pyridine, N,N-dimethylaminopyridine, or the like; alkali metal carbonates, e.g., sodium carbonate, potassium carbonate, or the like; alkali metal bicarbonates, e.g., sodium bicarbonate, potassium bicarbonate, or the like; alkali metal hydroxides, e.g., sodium hydroxide, potassium hydroxide, or the like; ammonia; resins bound to ions such as sodium, potassium, lithium, calcium, magnesium, or the like; any mixtures thereof; or any other suitable bases; either alone or as their aqueous solutions.

Step a) may be carried out in the presence of one or more suitable catalyst. Suitable catalysts, which may be used in step b) include, but are not limited to, triethylamine, pyridine, diisopropylethylamine, 1,8-diazabicyclo[5.4.0]undec-7-ene (DBU), 1,4-diazabicyclo[2.2.2]octane (DABCO), 1-methylmorpholine, 1-methyl piperidine, 1,5-diazabicyclo[4.3.0]non-5-ene, N,N-dimethylpiperazine, N,N-dimethyl aniline, 4-(dimethylamino)-pyridine (DMAP), hexamethylenetetramine (HMTA), tetra methylethylenediamine (TMEDA), collidine, 2,3,5,6-tetramethylpyridine (TEMP), or the like. Step a) may be carried out in presence of one or more suitable coupling agents. Suitable coupling agents, which may be used include, but are not limited to, N-hydroxy benzotriazole (HOBT), 4,5-dicyanoimidazole, dicyclohexylcarbodiimide (DCC), dicyclopentylcarbodiimide, diisopropylcarbodiimide, 1-ethyl-3-(3-dimethylamino propyl)carbodiimide hydrochloride, 1,1′-carbonyldiimidazole, cyclohexylisopropyl carbodiimide (CIC), bis[[4-(2,2-dimethyl-1,3-dioxolyl)]-methyl]carbodiimide, N,N′-bis(2-oxo-3-oxazolidinyl)-phosphinic chloride (BOP-CI), an acid chloride, ethyl chloroformate, or the like.

Step a) may be carried out in one or more suitable solvents. Suitable solvents, which may be used include, but are not limited to, an alcohol, e.g., methanol, ethanol, isopropyl alcohol, 1-propanol, 1-butanol, 2-butanol, or the like; a ketone, e.g., acetone, ethyl methyl ketone, methyl isobutyl ketone, or the like; a hydrocarbon, e.g., toluene, xylene, hexanes, heptanes, cyclohexane, or the like; a halogenated hydrocarbon, e.g., dichloromethane, ethylene dichloride, chloroform, or the like; an ester, e.g., ethyl acetate, n-propyl acetate, n-butyl acetate, t-butyl acetate, or the like; an ether, e.g., diethyl ether, diisopropyl ether, methyl t-butyl ether, tetrahydrofuran, dioxane, or the like; a polar aprotic solvent, e.g., N,N-dimethylformamide, N,N-dimethylacetamide, dimethylsulfoxide, sulfolane, N-methylpyrrolidone, or the like; a nitrile, e.g., acetonitrile, propionitrile, or the like; water; or any mixtures thereof.

Suitable temperatures for the reaction of step a) are less than about 100° C., less than about 80° C., less than about 60° C., less than about 40° C., less than about 20° C., less than about 0° C., or any other suitable temperatures.

Suitable times for completing the reaction in step a) depend on the temperature and other conditions, and may be generally less than about 30 hours, less than about 20 hours, less than about 10 hours, less than about 5 hours, less than about 2 hours, less than about 1 hour, or any other suitable times. Longer times also are suitable.

The product formed in step a) may be optionally recovered as a solid by conventional methods, including decantation, centrifugation, gravity filtration, suction filtration, or other techniques known in the art for the recovery of solids. The resulting solid may be optionally further dried. Drying may be suitably carried out using a tray dryer, vacuum oven, air oven, fluidized bed dryer, spin flash dryer, flash dryer, or the like, at atmospheric pressure or under reduced pressure. Drying may be carried out at temperatures less than about 100° C., less than about 60° C., less than about 40° C., or any other suitable temperatures, at atmospheric pressure or under reduced pressure, and in the presence or absence of an inert atmosphere such as nitrogen, argon, neon, or helium. The drying may be carried out for any desired time periods to achieve the desired quality of the product, such as, for example, about 1 to about 15 hours, or longer. Optionally, the product of step a) may be directly used in step b) without further isolation or after conventional work-up, such as, for example, quenching the reaction mixture with a quenching agent and extracting the product into a solvent.

Step b) involves partially hydrolyzing a compound of Formula VII to obtain a compound of Formula VIII. Step b) may be carried out in the presence of one or more suitable reagent such as a base, resin or any other suitable reagent. Suitable bases that may be used in step b) include but are not limited to: inorganic bases, such as, for example, ammonia, sodium hydroxide, potassium hydroxide, sodium methoxide, potassium t-butoxide, sodium t-butoxide, sodium carbonate, potassium carbonate, sodium bicarbonate, potassium bicarbonate, or the like; organic bases, such as, for example, triethylamine, n-propylamine, pyridine, N-methylmorpholine, diisopropylamine or diisopropylethylamine, or the like; resins including, but not limited to, ion exchange resins, such as resins bound to metal ions, including lithium, sodium, potassium, or the like; any mixtures thereof; or any other suitable reagents; either alone or as their aqueous solutions.

Optionally, step b) may be carried out in one or more suitable solvents. Suitable solvents include, but are not limited to: hydrocarbon solvents, including toluene, xylene, hexanes, heptanes, cyclohexane, or the like; halogenated hydrocarbon solvents, including dichloromethane, ethylene dichloride, chloroform, or the like; alcohol solvents, including methanol, ethanol, isopropyl alcohol, 1-propanol, 1-butanol, 2-butanol, or the like; ketone solvents, including acetone, ethyl methyl ketone, methyl isobutyl ketone, or the like; ester solvents, including ethyl acetate, n-propyl acetate, n-butyl acetate, t-butyl acetate, or the like; ether solvents, including diethyl ether, diisopropyl ether, methyl t-butyl ether, tetrahydrofuran, dioxane, or the like; polar aprotic solvents, including N,N-dimethylformamide, N,N-dimethylacetamide, dimethylsulfoxide, sulfolane, N-methylpyrrolidone, or the like; nitrile solvents, including acetonitrile, propionitrile, or the like; water; any mixtures thereof; or any other suitable solvents.

Suitable temperatures for the reaction of step b) may be less than about 150° C., less than about 120° C., less than about 100° C., less than about 80° C., less than about 60° C., less than about 40° C., or any other suitable temperatures. Suitable times for completion of hydrolysis in step b) depend on the temperature and other conditions and may be generally less than about 30 hours, less than about 24 hours, less than about 20 hours, less than about 10 hours, less than about 5 hours, less than about 1 hour, less than about 30 minutes, or any other suitable times.

Optionally, step b) may afford valganciclovir and pharmaceutically acceptable salts thereof by employing a suitable combination of reagent and solvent, including, for example, employing a base which performs partial hydrolysis of compound of Formula VII, as well as removal of protecting group P₁.

The product obtained in step b) may be recovered by conventional methods including decantation, centrifugation, gravity filtration, suction filtration, or other techniques known in the art. The resulting compound may be in the form of a residue or a solid. When it is a solid, it may be crystalline or amorphous in nature. When it is in the form of a solid, it may be optionally further dried. Drying may be suitably carried out using a tray dryer, vacuum oven, air oven, fluidized bed dryer, spin flash dryer, flash dryer, or the like, at atmospheric pressure or under reduced pressure. Drying may be carried out at temperatures less than about 150° C., less than about 120° C., less than about 100° C., less than about 60° C., less than about 40° C., or any other suitable temperatures, at atmospheric pressure or under reduced pressure, and in the presence or absence of an inert atmosphere, such as nitrogen, argon, neon, or helium. The drying may be carried out for any desired time periods to achieve the desired quality of the product, such as, for example, about 1 to about 15 hours, or longer. The resulting solid may be crystalline or amorphous in nature.

Optionally, the product of step b) may be further purified one or more times by any suitable techniques known in the art. For example the product of step b) may be purified by precipitation, slurrying in a suitable solvent, or any other suitable techniques. The precipitation may be achieved by crystallization, such as by cooling a solution or by adding an anti-solvent to a solution of the product, or any other suitable methods known in the art. Anti-solvents are liquids in which valganciclovir or its salt is poorly soluble. Suitable anti-solvents include, but are not limited to: hydrocarbon solvents (e.g., hexanes, heptanes, cyclohexane, toluene, xylenes or the like); ether solvents (e.g., diethyl ether, diisopropyl ether, methyl t-butyl ether, or the like); or any other suitable anti-solvents.

Suitable solvents for purification include, but are not limited to: halogenated hydrocarbon solvents, including dichloromethane, ethylene dichloride, chloroform, or the like; alcohol solvents, including methanol, ethanol, 1-propanol, 2-propanol, 1-butanol, 2-butanol, or the like; ketone solvents, including acetone, ethyl methyl ketone, methyl isobutyl ketone, or the like; ester solvents, including ethyl acetate, n-propyl acetate, n-butyl acetate, t-butyl acetate, or the like; ether solvents, including diethyl ether, diisopropyl ether, methyl t-butyl ether, tetrahydrofuran, dioxane, or the like; polar aprotic solvents, including N,N-dimethylformamide, N,N-dimethylacetamide, dimethylsulfoxide, sulfolane, N-methylpyrrolidone, or the like; nitrile solvents, including acetonitrile, propionitrile, or the like; water; any mixtures thereof; or any other suitable solvents. Purification may be carried out at suitable temperatures less than about 150° C., less than about 120° C., less than about 100° C., less than about 80° C., less than about 60° C., less than about 40° C., or any other suitable temperatures. Suitable times for purification depend on the temperature and other conditions and may be generally less than about 30 hours, less than about 24 hours, less than about 20 hours, less than about 10 hours, less than about 5 hours, less than about 1 hour, less than about 30 minutes, or any other suitable times.

The product thus obtained may be recovered by conventional methods including decantation, centrifugation, gravity filtration, suction filtration, or other techniques known in the art. The resulting compound may be in the form of a residue or a solid. When it is in the form of a solid, it may be optionally further dried. Drying may be suitably carried out using a tray dryer, vacuum oven, air oven, fluidized bed dryer, spin flash dryer, flash dryer, or the like, at atmospheric pressure or under reduced pressure. Drying may be carried out at temperatures less than about 150° C., less than about 120° C., less than about 100° C., less than about 60° C., less than about 40° C., or any other suitable temperatures, at atmospheric pressure or under reduced pressure, and in the presence or absence of an inert atmosphere, such as nitrogen, argon, neon, or helium. The drying may be carried out for any desired time periods to achieve the desired quality of the product, such as, for example, about 1 to about 15 hours, or longer.

Step c) involves converting a compound of Formula VIII to valganciclovir or a salt thereof. Step c) may be carried out using any suitable deprotection technique, including, for example, catalytic hydrogenation using hydrogen gas in the presence of a metal, including Raney nickel, palladium on carbon, or the like; or hydrolysis using an acid or base; or any other suitable deprotection agents known in the art. Optionally, catalytic hydrogenation may be carried out in the presence of one or more suitable reagent. Suitable reagents that may be used include, but are not limited to, acids, bases, resins, or mixtures thereof, either alone or as their solutions in water, organic solvents or their mixtures. Suitable acids that may be used in step c) include but are not limited to: organic acids, including acetic acid, formic acid, propionic acid, butyric acid, isobutyric acid, fumaric acid, oxalic acid, tartaric acid, citric acid, or the like; and inorganic acids, including hydrochloric acid, hydrobromic acid, hydroiodic acid, nitric acid, sulfuric acid, phosphoric acid, methanesulfonic acid, p-toluenesulfonic acid, or the like. Suitable bases that may be used in step c) include but are not limited to: inorganic bases, including ammonia, sodium hydroxide, potassium hydroxide, sodium methoxide, potassium t-butoxide, sodium t-butoxide, sodium carbonate, potassium carbonate, sodium bicarbonate, potassium bicarbonate, or the like; and organic bases, such as triethylamine, pyridine, N-methylmorpholine, diisopropylamine, diisopropylethylamine, or the like. Suitable resins that may be used in step c) include, but are not limited to, ion exchange resins, such as: resins bound to metal ions, including lithium, sodium, potassium, or the like; and resins bound to acids, including phosphoric, sulfonic, methanesulfonic, p-toluenesulfonic, or the like.

Optionally, the deprotection in step c) may be carried out in the presence of one or more suitable solvents. Suitable solvents that may be used in step c) include, for example: alcohol solvents, e.g., methanol, ethanol, isopropyl alcohol, 1-propanol, 1-butanol, 2-butanol, or the like; ketone solvents, e.g., acetone, ethyl methyl ketone, methyl isobutyl ketone, or the like; hydrocarbon solvents, e.g., toluene, xylene, hexanes, heptanes, cyclohexane, or the like; halogenated hydrocarbon solvents, e.g., dichloromethane, ethylene dichloride, chloroform, or the like; ester solvents, e.g., ethyl acetate, n-propyl acetate, n-butyl acetate, t-butyl acetate, or the like; ether solvents, e.g., diethyl ether, diisopropyl ether, methyl t-butyl ether, tetrahydrofuran, dioxane, or the like; polar aprotic solvents, e.g., N,N-dimethylformamide, N,N-dimethylacetamide, dimethylsulfoxide, sulfolane, N-methylpyrrolidone, or the like; nitrile solvents, e.g., acetonitrile, propionitrile, or the like; water; or any mixtures thereof.

Step c) may be carried out at suitable temperatures less than about 150° C., less than about 100° C., less than about 60° C., less than about 40° C., or any other suitable temperatures. Optionally, step c) may be carried out at atmospheric pressure or under pressure. Suitable pressures that may be used are less than about 10 kg/cm², less than about 5 kg/cm², less than about 3 kg/cm², less than about 1 kg/cm², or any other suitable pressures. Suitable times for completing step c) depend on temperature and other conditions and may be generally less than about 15 hours, less than about 10 hours, less than about 5 hours, less than about 2 hours, less than about 30 minutes, or any other suitable times.

Optionally, the product formed in step c) after deprotection, which comprises valganciclovir or a salt thereof, may be further treated with suitable reagents before or after conventional work-up process or after isolation of the compound as described in International Application No. PCT/US2009/058397. Suitable reagents that may be used for the treatment include, but are not limited to, phosphines, resins, or mixtures thereof, or any other suitable reagents. Suitable phosphines that may be used include, but are not limited to, triphenylphosphine, tri-n-butylphosphine, or the like. Suitable resins that may be used include, but are not limited to, ion exchange resins, including resins bound to metal ions, such as lithium, sodium, potassium, or the like, and resins bound to acids, such as phosphoric, sulfonic, methanesulfonic, p-toluenesulfonic, or the like.

Optionally, one or more suitable solvents may be used in the treatment. Suitable solvents that may be used include, for example: alcohol solvents, e.g., methanol, ethanol, isopropyl alcohol, 1-propanol, 1-butanol, 2-butanol, or the like; ketone solvents, e.g., acetone, ethyl methyl ketone, methyl isobutyl ketone, or the like; hydrocarbon solvents, e.g., toluene, xylene, hexanes, heptanes, cyclohexane, or the like; halogenated hydrocarbon solvents, e.g., dichloromethane, ethylene dichloride, chloroform, or the like; ester solvents, e.g., ethyl acetate, n-propyl acetate, n-butyl acetate, t-butyl acetate, or the like; ether solvents, e.g., diethyl ether, diisopropyl ether, methyl t-butyl ether, tetrahydrofuran, dioxane, or the like; polar aprotic solvents, e.g., N,N-dimethylformamide, N,N-dimethylacetamide, dimethylsulfoxide, sulfolane, N-methylpyrrolidone, or the like; nitrile solvents, e.g., acetonitrile, propionitrile, or the like; water; or any mixtures thereof.

The treatment may be carried out at suitable temperatures less than about 150° C., less than about 100° C., less than about 60° C., less than about 40° C., or any other suitable temperatures, at atmospheric pressure or elevated pressures. Suitable pressures are less than about 10 kg/cm², less than about 5 kg/cm², less than about 3 kg/cm², less than about 1 kg/cm², or any other suitable pressures. Suitable times for completing the treatment depend on temperature and other conditions and may be generally less than about 15 hours, less than about 10 hours, less than about 5 hours, less than about 2 hours, less than about 30 minutes, or any other suitable times.

The product obtained after said treatment may be recovered using conventional methods including decantation, centrifugation, gravity filtration, suction filtration, or other techniques known in the art. The resulting compound may be in the form of a residue or a solid. When it is in the form of a solid, it may be optionally further dried. Drying may be suitably carried out using a tray dryer, vacuum oven, air oven, fluidized bed dryer, spin flash dryer, flash dryer, or the like, at atmospheric pressure or under reduced pressure. Drying may be carried out at temperatures less than about 150° C., less than about 120° C., less than about 100° C., less than about 60° C., less than about 40° C., or any other suitable temperatures, at atmospheric pressure or under reduced pressure, and in the presence or absence of an inert atmosphere, such as nitrogen, argon, neon, or helium. The drying may be carried out for any desired time periods to achieve the desired quality of the product, such as, for example, about 1 to about 15 hours, or longer.

Optionally, the product obtained after the treatment, which comprises valganciclovir or a salt thereof, may be further purified using purification techniques known in the art, for example using column chromatography or various types of isolation methods including precipitation, adding an anti-solvent to a solution, or the like, in order to achieve a diastereomeric ratio of valganciclovir or its salt in the range of (45:55) to (55:45). Suitable solvents that may be used for purification include, but are not limited to: alcohol solvents, e.g., methanol, ethanol, isopropyl alcohol, 1-propanol, 1-butanol, 2-butanol, or the like; ketone solvents, e.g., acetone, ethyl methyl ketone, methyl isobutyl ketone, or the like; ester solvents, e.g., ethyl acetate, n-propyl acetate, n-butyl acetate, t-butyl acetate, or the like; polar aprotic solvents, e.g., N,N-dimethylformamide, N,N-dimethylacetamide, dimethylsulfoxide, sulfolane, N-methylpyrrolidone, or the like; water; any mixtures thereof in varying proportions; or any other suitable solvents.

The purification may be carried out at suitable temperatures less than about 150° C., less than about 100° C., less than about 60° C., less than about 40° C., or any other suitable temperatures. Suitable times for completing the purification depend on temperature and other conditions and may be generally less than about 15 hours, less than about 10 hours, less than about 5 hours, less than about 2 hours, less than about 30 minutes, or any other suitable times.

The product thus obtained may be recovered using conventional methods including decantation, centrifugation, gravity filtration, suction filtration, or other techniques known in the art. The resulting compound may be in the form of a residue or a solid. When it is in the form of a solid, it may be in the form of a crystalline compound, a solvate, an amorphous compound, or a mixture thereof. The solid may be optionally further dried. Drying may be suitably carried out using a tray dryer, vacuum oven, air oven, fluidized bed dryer, spin flash dryer, flash dryer, or the like, at atmospheric pressure or under reduced pressure. Drying may be carried out at temperatures less than about 150° C., less than about 120° C., less than about 100° C., less than about 60° C., less than about 40° C., or any other suitable temperatures, at atmospheric pressure or under reduced pressure, and in the presence or absence of an inert atmosphere, such as nitrogen, argon, neon, or helium. The drying may be carried out for desired time periods to achieve the desired quality of the product, such as, for example, about 1 to about 15 hours, or longer.

Valganciclovir or a salt thereof thus obtained may be optionally milled or micronized before or after drying of the product to get desired particle size. Milling or micronization may be performed by using techniques including; without limitation; milling using mills such as ball mill, roller mill, hammer mill, jet mill, air jet mill, co mill, multi mill or any other conventional technique. The pressures that may be used for milling or micronization are less than about 20 kg/cm², less than about 10 kg/cm², less than about 8 kg/cm², less than about 6 kg/cm², less than about 4 kg/cm², or less than about 3 kg/cm².

Valganciclovir or its salt of the present application is substantially free of metal contaminants. The metal contaminants can include, but are not limited to, palladium, nickel, cobalt, or any other metals. “Substantially free of metal contaminants” as used herein unless otherwise defined refers to a metal content less than about 50 ppm (parts per million), less than about 40 ppm, less than about 30 ppm, less than about 20 ppm, less than about 10 ppm, less than about 5 ppm, less than about 2 ppm, or less than about 1 ppm. All tautomeric forms of the compounds within the present application are within the scope of the present invention. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C— or ¹⁴C-enriched carbon are within the scope of this invention.

### DEFINITIONS

The following definitions are used in connection with the present application unless the context indicates otherwise. DMSO is dimethylsulfoxide and HPLC is high-pressure liquid chromatography. The term “reacting” is intended to represent bringing the chemical reactants together under conditions such to cause the chemical reaction indicated to take place.

An “alcohol solvent” is an organic solvent containing a carbon bound to a hydroxyl group. “Alcohol solvents” include but are not limited to methanol, ethanol, 2-nitroethanol, 2-fluoroethanol, 2,2,2-trifluoroethanol, hexafluoroisopropyl alcohol, ethylene glycol, 1-propanol, 2-propanol (isopropyl alcohol), 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, glycerol, C₁₋₆alcohols, or the like.

“Amine-protecting group” refers to a radical when attached to a nitrogen atom in a target molecule is capable of surviving subsequent chemical reactions applied to the target molecule i.e. hydrogenation, reaction with acylating agents, alkylation etc. The amine-protecting group can later be removed. Amine protecting groups include, but are not limited to, fluorenylmethoxycarbonyl (FMOC), tert-butoxycarbonyl (t-BOC), benzyloxycarbonyl (Z), those of the acyl type (e.g., formyl, benzoyl, trifluoroacetyl, p-tosyl, aryl- and alkylphosphoryl, phenyl- and benzylsulfonyl, o-nitrophenylsulfenyl, o-nitrophenoxyacetyl), and of the urethane type (e.g. tosyloxyalkyloxy-, cyclopentyloxy-, cyclohexyloxy-, 1,1-dimethylpropyloxy, 2-(p-biphenyl)-2-propyloxy- and benzylthiocarbonyl). Amine-protecting groups are made using a reactive agent capable of transferring an amine-protecting group to a nitrogen atom in the target molecule. Examples of an amine-protecting agent include, but are not limited to, C₁-C₆ aliphatic acid chlorides or anhydrides, C₆-C₁₄arylcarboxylic acid chlorides or anhydrides, t-butyl chloroformate, di-tert-butyl dicarbonate, butoxycarbonyloxyimino-2-phenylacetonitrile, t-butoxycarbonyl azide, t-butyl fluoroformate, fluorenylmethoxycarbonyl chloride, fluorenylmethoxycarbonyl azide, fluorenylmethoxycarbonyl benzotriazol-1-yl, (9-fluorenylmethoxycarbonyl)succinimidyl carbonate, fluorenylmethoxycarbonyl pentafluorophexoxide, trichloroacetyl chloride, methyl-, ethyl-, trichloromethyl-chloroformate, and other amine protecting agents known in the art. Examples of such known amine-protecting agents are found in pages 385-397 of T. W. Green, P. G. M. Wuts, “Protective Groups in Organic Synthesis, Second Edition”, Wiley-Interscience, New York, 1991.

An “anti-solvent” as used herein refers to a liquid in which valganciclovir or a salt thereof is less soluble or poorly soluble. Suitable anti-solvents include: hydrocarbon solvents, e.g., hexanes, heptanes, cyclohexane, toluene, xylenes, or the like; ether solvents, e.g., diethyl ether, diisopropyl ether, methyl t-butyl ether, or the like; or any other suitable anti-solvents.

An “ester solvent” is an organic solvent containing a carboxyl group —(C═O)—O-bonded to two other carbon atoms. “Ester solvents” include, but are not limited to, ethyl acetate, n-propyl acetate, n-butyl acetate, isobutyl acetate, t-butyl acetate, ethyl formate, methyl acetate, methyl propanoate, ethyl propanoate, methyl butanoate, ethyl butanoate, C₃₋₆esters, or the like.

An “ether solvent” is an organic solvent containing an oxygen atom —O— bonded to two other carbon atoms. “Ether solvents” include but are not limited to diethyl ether, diisopropyl ether, methyl t-butyl ether, glyme, diglyme, tetrahydrofuran, 1,4-dioxane, dibutyl ether, dimethylfuran, 2-methoxyethanol, 2-ethoxyethanol, anisole, C₂₋₆ethers, or the like.

A “halogenated hydrocarbon solvent” is an organic solvent containing a carbon bound to a halogen. “Halogenated hydrocarbon solvents” include, but are not limited to, dichloromethane, 1,2-dichloroethane, trichloroethylene, perchloroethylene, 1,1,1-trichloroethane, 1,1,2-trichloroethane, chloroform, carbon tetrachloride, or the like.

“Hydrocarbon solvent” refers to a liquid hydrocarbon, which may be linear, branched, or cyclic. It may be saturated, unsaturated, or aromatic. It is capable of dissolving a solute to form a uniformly dispersed solution. Examples of a hydrocarbon solvent include, but are not limited to, n-pentane, isopentane, neopentane, n-hexane, isohexane, 3-methylpentane, 2,3-dimethylbutane, neohexane, n-heptane, isoheptane, 3-methylhexane, neoheptane, 2,3-dimethylpentane, 2,4-dimethylpentane, 3,3-dimethylpentane, 3-ethylpentane, 2,2,3-trimethylbutane, n-octane, isooctane, 3-methylheptane, neooctane, C₅-C₈aliphatic hydrocarbons, cyclohexane, methylcyclohexane, ligroin, petroleum ethers, benzene, toluene, ethylbenzene, m-xylene, o-xylene, p-xylene, indane, naphthalene, tetralin, trimethylbenzene, C₆-C₁₀aromatic hydrocarbons, or mixtures thereof.

A “ketone solvent” is an organic solvent containing a carbonyl group —(C═O)— bonded to two other carbon atoms. “Ketone solvents” include, but are not limited to, acetone, ethyl methyl ketone, diethyl ketone, methyl isobutyl ketone, C₃₋₆ketones, or the like.

A “nitrile solvent” is an organic solvent containing a cyano —(C≡N) bonded to another carbon atom. “Nitrile solvents” include, but are not limited to, acetonitrile, propionitrile, C₂₋₆nitriles, or the like.

A “polar aprotic solvent” has a dielectric constant greater than 15 and is at least one selected from the group consisting of amide-based organic solvents, such as hexamethyl phosphoramide (HMPA), and hexamethyl phosphorus triamide (HMPT); nitro-based organic solvents, such as nitromethane, nitroethane, nitropropane, and nitrobenzene; ester-based organic solvents, such as γ-butyrolactone, ethylene carbonate, propylene carbonate, butylene carbonate, dimethyl carbonate, and propiolactone; pyridine-based organic solvents, such as pyridine and picoline; sulfone-based solvents, such as dimethyl sulfone, diethyl sulfone, diisopropylsulfone, 2-methylsulfolane, 3-methylsulfolane, 2,4-dimethyl-sulfolane, 3,4-dimethyl sulfolane, 3-sulfolene, and sulfolane; and nitrile-based organic solvents, such as acetonitrile, propionitrile, and benzonitrile. These organic solvents may be used alone or two or more of these may be combined appropriately.

“Protecting group” means a chemical group that (a) preserves a reactive group from participating in an undesirable chemical reaction, and (b) can be removed after protection of the reactive group is no longer required. For example, a benzyl group is a protecting group for a primary hydroxyl function. “Amine-protecting group” means a protecting group that preserves a reactive amine group that otherwise would be modified by certain chemical reactions. Useful amine protecting groups include, but are not limited to: benzyloxycarbonyl (Cbz), tert-butyloxycarbonyl (BOC), 9-fluorenylmethoxycarbonyl (FMOC), trifluoroacetyl, benzyl, trityl, formyl, or the like.

“Suitable coupling agent” refers to a compound, molecule, or substance, capable of activating carboxylic acids with respect to nucleophilic attack. In some embodiments, the suitable coupling agents are capable of activating carboxylic acids where the attacking nucleophile is an amine or alcohol, resulting in amide or ester formation. Non-limiting examples of such suitable coupling agents include carbodiimide compounds (e.g. N,N′-dicyclohexylcarbodiimide (DCC), N,N′-diisopropylcarbodiimide (DIC), 1-ethyl-3-(3-dimethylaminopropyl)carbodiimide hydrochloride (EDC.HCl), or the like). Carbodiimide compounds may be either used alone or in combination with HOAt, HOBt, or HODhbt. Further examples of suitable coupling agents include alkyl chloroformate compounds (e.g. ethyl chloroformate, isobutyl chloroformate, or the like) that are generally used with a tertiary amine like triethyl amine, diethyl azodicarboxylate (DEAD) with triphenylphosphine (the Mitsunobu reaction), various chlorosilanes, chlorosulfonyl isocyanate, N,N′-carbonyldiimidazole (CDI), phosphonium reagents (e.g. BOP, AOP, PyBOP, PyAOP, BroP, PyBroP, CF₃—NO₂-PyBOP or the like), in situ acid fluoride generators (e.g. TFFH, BTFFH, DAST, cyanuric fluoride, or the like), aminium reagents (e.g. HBTU, HATU, HBPyU, HAPyU, or the like) phosphinyls (e.g. DPPA, DEPC, or the like), pentafluorophenyl active ester generators (e.g. PfTU, PfPyU, FDPP, PFP-trifluoroacetate, FPFOH plus DCC, or the like), mixed carbon anhydrides (e.g. EEDQ, IIDQ, or the like), CIP, and BOP-CI.

All percentages and ratios used herein are by weight of the total composition and all measurements made are at about 25° C. and about atmospheric pressure, unless otherwise designated. All temperatures are in degrees Celsius unless specified otherwise. As used herein, “comprising” (open ended) means the elements recited, or their equivalent in structure or function, plus any other element or elements that are not recited. The terms “having” and “including” are also to be construed as open ended. All ranges recited herein include the endpoints, including those that recite a range “between” two values. Terms such as “about,” “generally,” “substantially,” or the like are to be construed as modifying a term or value such that it is not an absolute. Such terms will be defined by the circumstances and the terms that they modify as those terms are understood by those of skill in the art. This includes the degree of expected experimental error, technique error, and instrument error for a given technique used to measure a value.

Certain specific aspects and embodiments of the present disclosure will be explained in more detail with reference to the following examples, which are provided only for purposes of illustration and should not be construed as limiting the scope of the disclosure in any manner. Reasonable variations of the described procedures are intended to be within the scope of the present disclosure.

### EXAMPLES

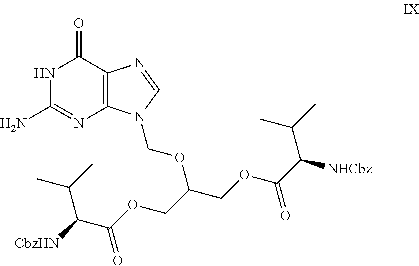

### Example 1

PREPARATION OF BIS-(Cbz-L-VALYL)-ESTER OF GANCICLOVIR (FORMULA IX). To a solution of ganciclovir (10.0 g) in DMSO (80 mL) is added Cbz-L-valine (29.5 g), 4-(dimethylamino)-pyridine (DMAP) (400 mg), and dicyclohexylcarbodiimide (DCC) (24.2 g) at 25-30° C. The mass is maintained at 25-30° C. for 5-6 hours. The mass is filtered and washed with DMSO (20 mL). The filtrate is added to 10% sodium chloride solution (200 mL) and ethyl acetate (70 mL). The organic layer is separated and aqueous layer is washed with ethyl acetate (30 mL). The combined organic layer is washed with 20% sodium chloride solution (100 mL). Cyclohexane (300 mL) is added to the organic layer and the mixture is maintained for 12-14 hours at 25-30° C. The formed solid is collected by filtration, washed with cyclohexane (50 mL), and dried to afford the title compound. Yield: 26.5 g. Purity by HPLC: 98.87%; mono-(Cbz-L-valyl)-ester of Formula X: 0.58%.

### Example 2

PREPARATION OF BIS-(Cbz-L-VALYL)-ESTER OF GANCICLOVIR (FORMULA IX). To a solution of ganciclovir (10.0 g) in DMSO (175 mL) is added Cbz-L-valine (39.3 g), 4-(dimethylamino)-pyridine (DMAP) (100 mg) and dicyclohexylcarbodiimide (DCC, 36.3 g) at 25-30° C. The mass is maintained at 25-30° C. for 5-6 hours. The mass is filtered and washed with DMSO (20 mL). The filtrate is added to water (1200 mL) and ethyl acetate (100 mL) is added to produce a clear solution at 25-30° C. n-Hexane (500 mL) is added and the mass is maintained at 25-30° C. for 15-20 hours. The solid is collected by filtration, washed with n-hexane (50 mL), and dried to afford the title compound. Yield: 27.0 g. Purity by HPLC: 95.27%; mono-(Cbz-L-valyl)-ester of Formula X: 3.11%.

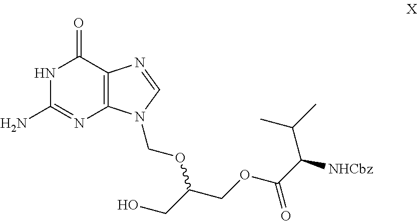

### Example 3

PREPARATION OF MONO-(Cbz-L-VALYL)-ESTER OF GANCICLOVIR (FORMULA X). To a solution of the bis-(Cbz-L-valyl)-ester of ganciclovir of Formula IX (25 g) in methanol (375 mL) is added diisopropylethylamine (33.3 g) at 25-30° C. The mixture is maintained for 15-20 hours at 25-30° C. Acetic acid (10.2 mL) is added and the mass is maintained for another 1-2 hours at 25-30° C. The solvent is evaporated and water (500 mL) and ethyl acetate (400 mL) are added, followed by refluxing for 30-60 minutes. The mass is maintained at 30-35° C. for 1-2 hours. The solid is collected by filtration, washed with ethyl acetate (70 mL), and is dried to afford the title compound. Yield: 7.5 g. Purity by HPLC: 96.5%; ganciclovir: 2.0%; bis-(Cbz-L-valyl)-ester of ganciclovir of Formula IX: 0.40%.

### Example 4

PREPARATION OF MONO-(Cbz-L-VALYL)-ESTER OF GANCICLOVIR (FORMULA X). To a solution of bis-(Cbz-L-valyl)-ester of ganciclovir of Formula IX (100 g) in methanol (200 mL) is added n-propylamine (12.28 g) at 25-30° C. The mixture is maintained for 24 hours at 25-30° C. Acetic acid (12.42 mL) is added, followed by addition of water (1.0 L) at 5° C. The solid is collected by filtration, washed with water (500 mL) and ethyl acetate (2×100 mL), and then dried to afford the crude title compound. The crude mono-(Cbz-L-valyl)-ester of ganciclovir of Formula X is further purified by repeated precipitation, involving dissolving in acetic acid followed by adding water and collecting the solid by filtration. The resulting compound is further purified by repeated crystallization from a mixture of ethyl acetate and water, to finally afford pure mono-(Cbz-L-valyl)-ester of ganciclovir of Formula X. Purity by HPLC: 97.65%; ganciclovir: 1.56%; bis-(Cbz-L-valyl)-ester of ganciclovir of Formula IX: 0.43%.

### Example 5

PREPARATION OF MONO-(Cbz-L-VALYL)-ESTER OF GANCICLOVIR (FORMULA X). To a solution of bis-(Cbz-L-valyl)-ester of ganciclovir of Formula IX (15 g) in methanol (225 mL) is added diisopropylamine (10.5 g) at 25-30° C. The mixture is maintained for 15-20 hours at 25-30° C. Acetic acid (8.24 mL) is added and the mass is maintained for another 1-2 hours at 25-30° C. The solvent is evaporated, and water (225 mL) and ethyl acetate (150 mL) are added, followed by refluxing for 30-60 minutes. The mass is maintained at 30-35° C. for 2-3 hours. The solid is collected by filtration, washed with water (100 mL), and dried to afford the crude title compound. The crude mono-(Cbz-L-valyl)-ester of ganciclovir of Formula X is further purified by repeated crystallization from a mixture of ethyl acetate and water to finally afford pure mono-(Cbz-L-valyl)-ester of ganciclovir of Formula X. Yield: 4.5 g. Purity by HPLC: 97.2%; ganciclovir: 0.97%; bis-(Cbz-L-valyl)-ester of ganciclovir of Formula IX: 1.25%.

### Example 6

PREPARATION OF VALGANCICLOVIR HYDROCHLORIDE SALT. To a solution of mono-(Cbz-L-valyl)-ester of ganciclovir of Formula X (6.5 g) in ethanol (208 mL) in an autoclave vessel is added aqueous HCl (1.62 mL) and 10% palladium on carbon (50% wet, 0.78 g), and a hydrogen pressure of 0.8-1 kg/cm² is maintained for 6-7 hours at 25-30° C. The mass is filtered and the collected solid is washed with ethanol (30 mL). The filtrate is evaporated below 40° C. to remove ethanol and the volume is adjusted to 10 mL with water. The filtrate is washed with toluene (3×13 mL) and 1-butanol (2×13 mL) at 25-30° C. Isopropyl alcohol (39 mL) is added to the mixture and stirred for 15 hours for solid formation at 25-30° C. Isopropyl alcohol (32.5 mL) is added and the mixture is stirred for 2-3 hours at 25-30° C. The mass is cooled and formed solid is collected by filtration at −10 to −15° C., washed with chilled isopropyl alcohol (13 mL), and dried under reduced pressure at 40-50° C. to afford the title compound. Yield: 3.6 g. Purity by HPLC: 96.25%; ganciclovir: 2.31%; bis-(Cbz-L-valyl)-ester of ganciclovir of Formula IX: 0.20%.

### Example 7

PREPARATION OF VALGANCICLOVIR HYDROCHLORIDE SALT. To a solution of mono-(Cbz-L-valyl)-ester of ganciclovir of Formula X (10 g) in methanol (320 mL) in an autoclave vessel is added aqueous HCl (2.33 mL) and 10% palladium on carbon (50% wet, 1.0 g), and a hydrogen pressure of 0.7-1 kg/cm² is maintained for 17-18 hours at 25-30° C. The mass is filtered and the collected solid is washed with methanol (46 mL). To the filtrate is added triphenylphosphine (25 mg) and charcoal. The reaction mass is maintained for 1-2 hours at 25-30° C. The mass is filtered and the collected solid is washed with methanol (30 mL). To the filtrate is added water (10 mL), the mixture is evaporated below 40° C. to remove methanol, and the volume is adjusted to 13.5 mL with water. The aqueous layer is washed with toluene (3×18 mL) and 1-butanol (2×18 mL) at 25-30° C. Isopropyl alcohol (100 mL) is added to the mixture and maintained for 15-18 hours for solid formation at 25-30° C. Cyclohexane (45 mL) is added to the reaction mass. Reaction mass is cooled and maintained for 1-2 hours at 0 to −15° C. The formed solid is collected by filtration at −10 to −15° C., washed with chilled isopropyl alcohol (18 mL), and dried under reduced pressure at 40-50° C. to afford the title compound. Yield: 5.0 g. Purity by HPLC: 98.24%; ganciclovir: 0.67%; methoxymethyl guanine: 0.09%; bis-(Cbz-L-valyl)-ester of ganciclovir of Formula IX: 0.33%; Pd content: 42 ppm.

### Example 8

PREPARATION OF BIS-(Cbz-L-VALYL)-ESTER OF GANCICLOVIR (FORMULA IX). Dicyclohexylcarbodiimide (DCC) (64.6 g) solution (DCC (64.6 kg) in DMSO (40 mL) is slowly added to a reaction mixture containing 9-((2-hydroxy-1-(hydroxymethyl)ethoxy)methyl) guanine (20 g), Cbz-L-valine (69 g), 4-(dimethylamino)-pyridine (DMAP) (0.8 g), and DMSO (140 mL) at 18-25° C. and the reaction mixture maintained at 18-25° C. for 30 minutes. The reaction mixture temperature is increased to 28° C. and stirred for 6 hours. The mass is filtered and the collected solid washed with DMSO (40 mL). The filtrate is added to a mixture of 5% sodium bicarbonate solution (500 mL) and ethyl acetate (140 mL) at 25-30° C. and stirred for 15 minutes. The organic layer is separated and the aqueous layer is extracted with ethyl acetate (60 mL). The combined organic layer is washed with 20% sodium chloride solution (2×200 mL). Cyclohexane (460 mL) is added to the organic layer at 25-35° C. and the mixture is maintained for 17 hours at 25-35° C. Reaction mixture is further cooled to 10-15° C. and maintained at 16° C. for 15 minutes. The separated solid is collected by filtration, washed with a pre-cooled (10-15° C.) cyclohexane (100 mL) and ethyl acetate (30 mL) mixture, and dried at 48° C. for 4 hours, under reduced pressure. Charged the dried compound and water (400 mL) into round bottom flask and stirred at 28° C. for 1 hour 30 minutes. Reaction mixture is filtered, the collected solid is washed with water (80 mL), and dried at 48° C. for 22 hours under reduced pressure to afford 47.8 g of the title compound. Purity by HPLC: 99.8%.

### Example 9

PREPARATION OF MONO-(Cbz-L-VALYL)-ESTER OF GANCICLOVIR (FORMULA X). Bis-(Cbz-L-valyl)-ester of ganciclovir of Formula IX (100 g) and methanol (200 mL) are charged into round bottom flask and stirred for 5 minutes. n-Propylamine (13.6 mL) is added to the reaction mass at 27° C. and maintained at 27° for 30 hours. Acetic acid (9.5 mL) is slowly added and the mass and cooled to 16° C. Acetic acid (390.5 mL) is added to the reaction mass at 15-20° C. and maintained reaction mass at 12° C. for 1 hour. The resultant reaction mass is slowly added to water (2000 mL) at 12° C. and maintained at 12° C. for 1 hour. The separated solid is collected by filtration, washed with pre-cooled (10-15° C.) water (1000 mL), and dried at 45° C. for 12 hours under reduced pressure. The obtained dry compound (80 g) and acetic acid (320 mL) is charged into round bottom flask and stirred for 1 hour. The resultant reaction mass is slowly added to water (1600 mL) at 12° C. and stirred for 1 hour. The separated solid is collected by filtration and washed with pre-cooled (10-15° C.) water (1600 mL). The obtained wet compound and ethyl acetate (600 mL) are charged into round bottom flask and stirred at 62° C. for 8 minutes. Reaction mass is cooled to 26° C. and stirred for 1 hour. The separated solid is collected by filtration and washed with ethyl acetate (60 mL). The obtained wet compound, ethyl acetate (750 mL), and water (22.5 mL) are charged into round bottom flask and stirred at 62° C. for 75 minutes. The reaction mass is cooled to 26° C. and stirred for 80 minutes. The separated solid is collected by filtration and washed with ethyl acetate (45 mL). The obtained wet compound, ethyl acetate (270 mL), and water (18 mL) are charged into round bottom flask and stirred at 62° C. for 1 hour. Reaction mass is cooled to 6° C. and stirred for 1 hour 30 minutes. The separated solid is collected by filtration and washed with ethyl acetate (36 mL). The obtained wet compound, ethyl acetate (216 mL), and water (14.4 mL) are charged into round bottom flask and stirred at 62° C. for 1 hour 30 minutes. Reaction mass is cooled to 26° C. and stirred for 70 minutes. The separated solid is collected by filtration, washed with ethyl acetate (28.8 mL), and dried at 45-50° C. for 10 hours under reduced pressure. Dry compound is milled and further dried at 45-50° C. for 24 hours to afford 28.0 g of the title compound. Purity by HPLC: 99.59%; ganciclovir: 0.37%; bis-(Cbz-L-valyl)-ester of ganciclovir of Formula IX: 0.22%.

### Example 10

PREPARATION OF VALGANCICLOVIR HYDROCHLORIDE SALT. To a solution of mono-(Cbz-L-valyl)-ester of ganciclovir of Formula X (10 g) in methanol (200 mL) in an autoclave vessel is added aqueous HCl (2.2 mL) and 10% palladium on carbon (50% wet, 1.0 g). A hydrogen pressure of 3.0-3.5 kg/cm² is maintained for 2 hours 30 minutes at 29° C. The mass is filtered and the collected solid is washed with methanol (20 mL). To the filtrate is added triphenylphosphine (20 mg) and charcoal. The reaction mass is maintained for 75 minutes at 28° C. The mass is filtered and the collected solid is washed with methanol (30 mL). The filtrate is evaporated below 40° C. to remove methanol and the water (10 mL) is added and stirred for 15 minutes. Isopropyl alcohol (200 mL) is added to the mixture at 3° C. and stirred for 3 hours. The separated solid is filtered and washed with isopropyl alcohol (20 mL). The obtained wet compound, water (15 mL) and n-propyl alcohol (30 mL) are charged into round bottom flask and stirred for 15 minutes. The resultant reaction mass is filtered and washed with mixture of water (5 mL) and n-propyl alcohol (10 mL). To the obtained filtrate n-propyl alcohol (160 mL) is added at 5° C. and stirred for 2 hours 10 minutes. The separated solid is filtered, washed with n-propyl alcohol (10 mL), and dried at 45° C. for 17 hours under reduced pressure to afford 5.7 g of the title compound.

Throughout this application, various publications are referenced. The disclosures of these publications in their entireties are hereby incorporated by reference into this application in order to more fully describe the state of the art as known to those skilled therein as of the date of the application described and claimed herein.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A processes comprising partially hydrolyzing in an alcohol solvent a compound of Formula VII to provide a compound of Formula VIII: 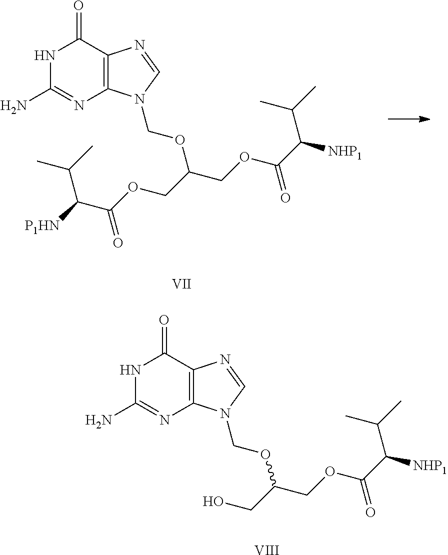
wherein P₁ is an amine-protecting group.

2. The process of claim 1, wherein the hydrolysis is carried out in the presence of triethylamine, n-propylamine, pyridine, N-methylmorpholine, diisopropyl amine or diisopropylethylamine.

3. The process of claim 2, wherein the hydrolysis is carried out in the presence of n-propylamine.

4. The process of claim 1, wherein the alcohol solvent is methanol.

5. The process of claim 1 further comprising reacting the compound of Formula III with a compound of Formula VI in presence of one or more suitable coupling agents and in one or more suitable solvents to provide a compound of Formula VII: 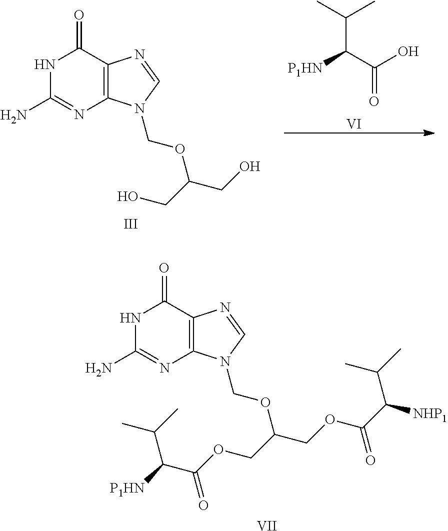
wherein P₁ is as defined above.

6. The process of claim 5, wherein P₁ is benzyloxycarbonyl.

7. The process of claim 5, wherein the coupling agent is N-hydroxybenzo triazole, 4,5-dicyanoimidazole, dicyclohexylcarbodiimide, dicyclopentylcarbodiimide, diisopropylcarbodiimide, 1-ethyl-3-(3-dimethylaminopropyl) carbodiimide hydrochloride, 1,1′-carbonyldiimidazole, cyclohexylisopropylcarbodiimide, bis[[4-(2,2-dimethyl-1,3-dioxolyl)]-methyl]carbodiimide, N,N′-bis(2-oxo-3-oxazolidinyl)-phosphinic chloride, an acid chloride, or ethyl chloroformate.

8. The process of claim 7, wherein the coupling agent is dicyclohexyl carbodiimide, dicyclopentylcarbodiimide, diisopropylcarbodiimide, 1-ethyl-3-(3-dimethylaminopropyl)carbodiimide hydrochloride, cyclohexylisopropylcarbodiimide, or bis[[4-(2,2-dimethyl-1,3-dioxolyl)]-methyl]carbodiimide.

9. The process of claim 8, wherein the coupling agent is dicyclohexyl carbodiimide.

10. The process of claim 5, further comprising reacting the compound of Formula III with a compound of Formula VI in presence of triethylamine, pyridine, diisopropylethylamine, 1,8-diazabicyclo[5.4.0]undec-7-ene (DBU), 1,4-diazabicyclo[2.2.2]octane (DABCO), 1-methylmorpholine, 1-methylpiperidine, 1,5-diazabicyclo[4.3.0]non-5-ene, N,N-dimethylpiperazine, N,N-dimethylaniline, 4-(dimethylamino)-pyridine (DMAP), hexamethylenetetramine (HMTA), tetramethylethylenediamine (TMEDA), collidine, or 2,3,5,6-tetramethylpyridine (TEMP).

11. The process of claim 10, wherein the reacting is in presence of 4-(dimethylamino)-pyridine (DMAP).

12. The process of claim 5, wherein the suitable solvents includes a polar aprotic solvent.

13. The process of claim 12, wherein the suitable solvent is dimethylsulfoxide.

14. The process of claim 1 further comprising converting a compound of Formula VIII to valganciclovir of formula I: 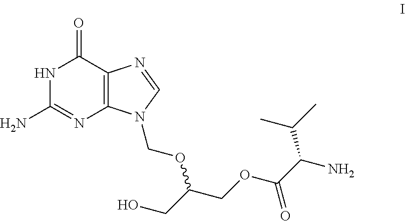
or a salt thereof.

15. The process of claim 14, wherein the converting is by catalytic hydrogenation using hydrogen gas in the presence of a metal and is carried out in the presence of one or more suitable solvents.

16. The process of claim 15, wherein the metal is palladium on carbon.

17. The process of claim 15, wherein the suitable solvent is an alcohol solvent.

18. The process of claim 17, wherein the alcohol solvent is methanol.

19. The process of claim 14, wherein the valganciclovir salt is the hydrochloride salt.
